(19) United States
(12) Patent Application Publication
(10) Pub. No.: US 2025/0381031 A1
(43) Pub. Date: Dec. 18, 2025

(54) Methods for Achieving Consistent Crimp Profile

(71) Applicant: St. Jude Medical, Cardiology Division, Inc., St. Paul, MN (US)

(72) Inventors: Jay Reimer, Shoreview, MN (US); Michael Shane Morrissey, St. Paul, MN (US)

(73) Assignee: St. Jude Medical, Cardiology Division, Inc., St. Paul, MN (US)

(21) Appl. No.: 19/087,013

(22) Filed: Mar. 21, 2025

Related U.S. Application Data
(60) Provisional application No. 63/658,965, filed on Jun. 12, 2024.

(51) Int. Cl. A61F 2/24

(52) U.S. Cl. CPC A61F 2/2427; A61F 2/2418; A61F 2/2496

(57) ABSTRACT

A method of implanting a prosthetic heart valve may include crimping the valve onto a delivery device from a larger initial diameter to a smaller crimped diameter. Before crimping, the prosthetic leaflets of the valve may be in an uncompressed condition, and after crimping, the prosthetic leaflets may be in a compressed condition. The prosthetic heart valve may be advanced through a patient's vasculature while crimped and then deployed into a valve annulus of the patient. After crimping the prosthetic heart valve form the initial diameter to the compressed diameter, the prosthetic heart valve may have a crimp magnitude of between 110% and 135%. The crimp magnitude may be calculated as a ratio of (i) a size of the prosthetic leaflets in the uncompressed condition to (ii) a total amount of available size that the prosthetic leaflets can occupy when the prosthetic leaflets are in the compressed condition.

### CROSS-REFERENCE TO RELATED APPLICATIONS

This application claims priority to the filing date of U.S. Provisional Patent Application No. 63/658,965, filed Jun. 12, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

The present disclosure addresses problems and limitations associated with the related art.

### SUMMARY OF THE DISCLOSURE

In some examples, balloon-expandable prosthetic heart valves are crimped to a small size, for example over an inflatable balloon, for transcatheter delivery into a patient. Although it is often desirable to crimp the prosthetic heart valve down to a small size for delivery, crimping the prosthetic heart valve to too small a size and/or for too long a time may damage the prosthetic heart valve and/or lead to other negative effects such as increased calcification upon implantation. However, by quantifying an amount by which the prosthetic heart valve has been crimped, over-crimping may be avoided. Further, in some examples, prosthetic heart valves are mounted to a delivery system during manufacturing, instead of immediately prior to use, with the prosthetic heart valve provided in a pre-crimped or pre-mounted configuration to the end user. In such examples, by quantifying an amount by which the prosthetic heart valve has been crimped, crimp uniformity among pre-crimped or pre-mounted prosthetic heart valves may be achieved, which may provide more predictability and consistency for end users.

According to one aspect of the disclosure, a method of implanting a prosthetic heart valve includes inserting the prosthetic heart valve into a crimper. The prosthetic heart valve may include a frame, an inner cuff disposed on a luminal surface of the frame, and a plurality of prosthetic tissue leaflets mounted within the frame, the prosthetic heart valve having an initial diameter when inserted into the crimper. While the prosthetic heart valve is inserted into the crimper, the prosthetic heart valve may be crimped onto a delivery device so that the prosthetic heart valve has a crimped diameter that is smaller than the initial diameter. When the prosthetic heart valve has the initial diameter, the prosthetic tissue leaflets may be in an uncompressed condition, and when the prosthetic heart valve has the crimped diameter, the prosthetic tissue leaflets may be in a compressed condition. The method may include advancing the prosthetic heart valve into a vasculature of a patient while the prosthetic heart valve has the crimped diameter, and deploying the prosthetic heart valve into a valve annulus of the patient. After crimping the prosthetic heart valve from the initial diameter to the compressed diameter, the prosthetic heart valve may have a crimp magnitude of between 110% and 135%. The crimp magnitude may be calculated as a ratio of (i) a size of the prosthetic tissue leaflets in the uncompressed condition to (ii) a total amount of available size that the prosthetic tissue leaflets can occupy when the prosthetic tissue leaflets are in the compressed condition. The size of the prosthetic tissue leaflets may be a volume of the prosthetic tissue leaflets, and the total amount of available size that the prosthetic tissue leaflets can occupy may be a total amount of available volume that the prosthetic tissue leaflets can occupy. The total amount of available volume that the prosthetic tissue leaflets can occupy may be calculated as a volume of a cylinder prescribed around an outer surface of the frame, less a volume of the frame within the cylinder, less a volume of the inner cuff within the cylinder, less a volume of delivery device components within the cylinder. The size of the prosthetic tissue leaflets may be a cross-sectional area of the prosthetic tissue leaflets, and the total amount of available size that the prosthetic tissue leaflets can occupy may be a total amount of available cross-sectional area that the prosthetic tissue leaflets can occupy. The size of the prosthetic tissue leaflets may be a thickness of the prosthetic tissue leaflets, and the total amount of available size that the prosthetic tissue leaflets can occupy may be a total amount of available thickness the prosthetic tissue leaflets can occupy. An amount of time between crimping the prosthetic heart valve and deploying the prosthetic heart valve may be under 45 minutes. The amount of time between crimping the prosthetic heart valve and deploying the prosthetic heart valve may be between 2 weeks and 6 months.

According to another aspect of the disclosure, a method of manufacturing a prosthetic heart valve may include inserting the prosthetic heart valve into a crimper. The prosthetic heart valve may include a frame, an inner cuff disposed on a luminal surface of the frame, and a plurality of prosthetic tissue leaflets mounted within the frame. The prosthetic heart valve may have an initial diameter when inserted into the crimper. While the prosthetic heart valve is inserted into the crimper, the prosthetic heart valve may be crimped onto a delivery device so that the prosthetic heart valve has a crimped diameter that is smaller than the initial diameter. When the prosthetic heart valve has the initial diameter, the prosthetic tissue leaflets may be in an uncompressed condition, and when the prosthetic heart valve has the crimped diameter, the prosthetic tissue leaflets may be in a compressed condition. A size of the prosthetic tissue leaflets in the uncompressed condition may be determined. A crimp magnitude of the prosthetic heart valve after crimping the prosthetic heart valve onto the delivery device may be determined. The crimp magnitude may be calculated as a ratio of (i) the determined size of the prosthetic tissue leaflets in the uncompressed condition to (ii) a total amount of available size that the prosthetic tissue leaflets can occupy when the prosthetic tissue leaflets are in the compressed condition. The crimp magnitude may be compared to a threshold acceptable crimp magnitude. Based on the comparison, the delivery device and the prosthetic heart valve may be packaged while the prosthetic heart valve is crimped onto the delivery device after determining from the comparison that the crimp magnitude does not exceed the threshold acceptable crimp magnitude. Alternatively, based on the comparison, the prosthetic heart valve may be discarded after determining from the comparison that the crimp magnitude does exceed the threshold acceptable crimp magnitude. The threshold acceptable crimp magnitude may be between 110% and 135%. The size of the prosthetic tissue leaflets may be a volume of the prosthetic tissue leaflets, and the total amount of available size that the prosthetic tissue leaflets can occupy may be a total amount of available volume that the prosthetic tissue leaflets can occupy. The total amount of available volume that the prosthetic tissue leaflets can occupy may be calculated as a volume of a cylinder prescribed around an outer surface of the frame, less a volume of the frame within the cylinder, less a volume of the inner cuff within the cylinder, less a volume of delivery device components within the cylinder. The crimp diameter may be a minimum diameter corresponding to a maximum crimping force applied by the crimper. The crimp diameter may be an equilibrium diameter, the equilibrium diameter being achieved after the prosthetic heart valve recoils from a minimum diameter corresponding to a maximum crimping force applied by the crimper. The crimper may be an automated or motorized crimper. Prior to crimping the prosthetic heart valve, a desired crimp diameter may be determined based on (i) the determined size of the prosthetic tissue leaflets in the uncompressed condition and (ii) the threshold acceptable crimp magnitude. The method may also include, after determining the desired crimp diameter, adjusting settings of the automated or motorized crimper to perform the crimping so that the prosthetic heart valve is crimped to the desired crimp diameter. After determining from the comparison that the crimp magnitude does not exceed the threshold acceptable crimp magnitude, the delivery device and the prosthetic heart valve may be packaged while the prosthetic heart valve is crimped onto the delivery device. After determining from the comparison that the crimp magnitude does exceed the threshold acceptable crimp magnitude, the prosthetic heart valve may be discarded.

According to a further example of the disclosure, a method of implanting a prosthetic heart valve includes inserting the prosthetic heart valve into a crimper. The prosthetic heart valve may include a frame, an inner cuff disposed on a luminal surface of the frame, and a plurality of prosthetic tissue leaflets mounted within the frame. The prosthetic heart valve may have an initial diameter when inserted into the crimper. While the prosthetic heart valve is inserted into the crimper, the prosthetic heart valve may be crimped onto a delivery device so that the prosthetic heart valve has a crimped diameter that is smaller than the initial diameter. When the prosthetic heart valve has the initial diameter, the prosthetic tissue leaflets may be in an uncompressed condition, and when the prosthetic heart valve has the crimped diameter, the prosthetic tissue leaflets may be in a compressed condition. A size of the prosthetic tissue leaflets in the uncompressed condition may be determined. A crimp magnitude of the prosthetic heart valve after crimping the prosthetic heart valve onto the delivery device may be determined. The crimp magnitude may be calculated as a ratio of (i) the determined size of the prosthetic tissue leaflets in the uncompressed condition to (ii) a total amount of available size that the prosthetic tissue leaflets can occupy when the prosthetic tissue leaflets are in the compressed condition, The crimp magnitude may be compared to a threshold acceptable crimp magnitude. Based on the comparison, the prosthetic heart valve may be implanted into a patient after determining from the comparison that the crimp magnitude does not exceed the threshold acceptable crimp magnitude. Alternatively, based on the comparison, the prosthetic heart valve may be discarded after determining from the comparison that the crimp magnitude does exceed the threshold acceptable crimp magnitude. The threshold acceptable crimp magnitude may be between 110% and 300%. The size of the prosthetic tissue leaflets may be a volume of the prosthetic tissue leaflets, and the total amount of available size that the prosthetic tissue leaflets can occupy may be a total amount of available volume that the prosthetic tissue leaflets can occupy. The total amount of available volume that the prosthetic tissue leaflets can occupy may be calculated as a volume of a cylinder prescribed around an outer surface of the frame, less a volume of the frame within the cylinder, less a volume of the inner cuff within the cylinder, less a volume of delivery device components within the cylinder. The crimp diameter may be a minimum diameter corresponding to a maximum crimping force applied by the crimper. The crimp diameter may be an equilibrium diameter, and the equilibrium diameter may be achieved after the prosthetic heart valve recoils from a minimum diameter corresponding to a maximum crimping force applied by the crimper. The crimper may be a manual crimper. Prior to crimping the prosthetic heart valve, a desired crimp diameter may be determined based on (i) the determined size of the prosthetic tissue leaflets in the uncompressed condition and (ii) the threshold acceptable crimp magnitude. After determining from the comparison that the crimp magnitude does not exceed the threshold acceptable crimp magnitude, the prosthetic heart valve may be implanted into the patient. One hour or less may elapse between crimping the prosthetic heart valve and implanting the prosthetic heart valve into the patient. Between two weeks and six months may elapse between crimping the prosthetic heart valve and implanting the prosthetic heart valve into the patient. The threshold acceptable crimp magnitude may be between 110% and 135%. After determining from the comparison that the crimp magnitude does exceed the threshold acceptable crimp magnitude, the prosthetic heart valve may be discarded.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12 is chart showing exemplary data comparing tissue calcification as a function of crimp magnitude.

FIG. 13 is chart showing exemplary data comparing tissue calcification as a function of time crimped.

FIG. 14 is a front view of the prosthetic heart valve of FIG. 1 in an example collapsed condition mounted on an example of a portion of a delivery system.

FIG. 15 is a side view of the prosthetic heart valve of FIG. 1 in an expanded condition.

FIG. 16 is a cross-section of the prosthetic heart valve of FIG. 15 in an example collapsed condition mounted on an example of a portion of a delivery system, taken along the section line 16-16 of FIG. 15.

FIG. 17 is a cross-section of half of the prosthetic heart valve of FIG. 15 in an example collapsed condition mounted on an example of a portion of a delivery system, taken along the section line 16-16 of FIG. 15.

FIG. 18 is a flow chart showing example steps of an example method of using crimp magnitude in a manufacturing process.

FIG. 19 is a flow chart showing example steps of an example method of using crimp magnitude in a manufacturing process.

FIG. 20 is a flow chart showing example steps of an example method of using crimp magnitude as part of an implantation procedure.

FIG. 21 is a flow chart showing example steps of an example method of using crimp magnitude as part of an implantation procedure.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

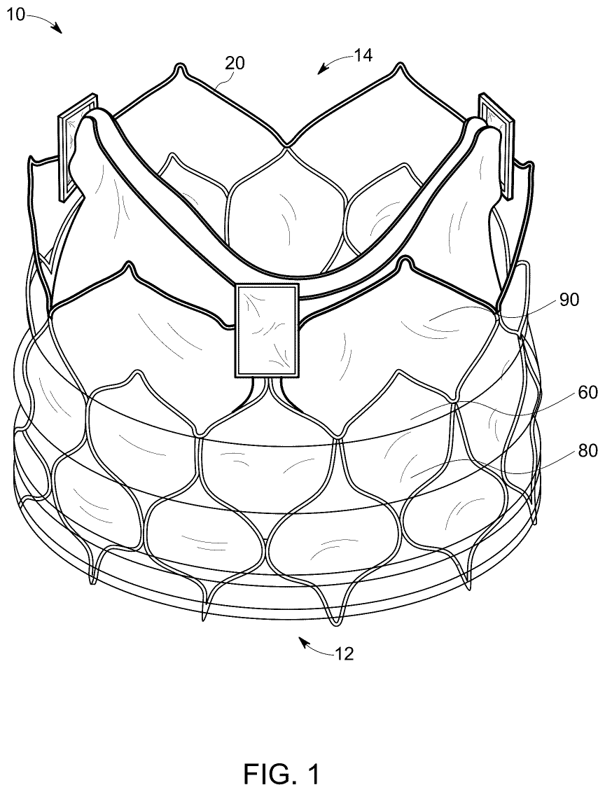

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

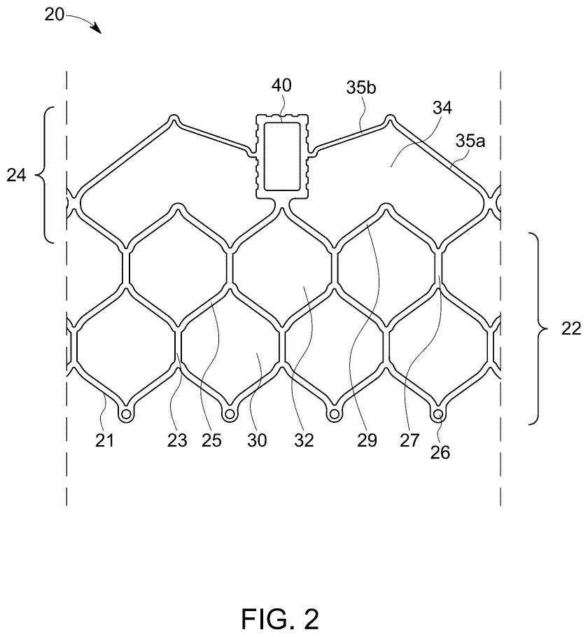

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g., fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Patent Application Publication No. 2025/0073023, the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g., struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

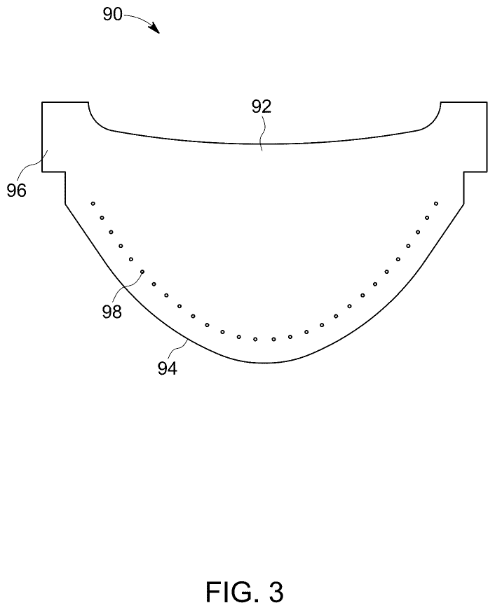

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g., via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

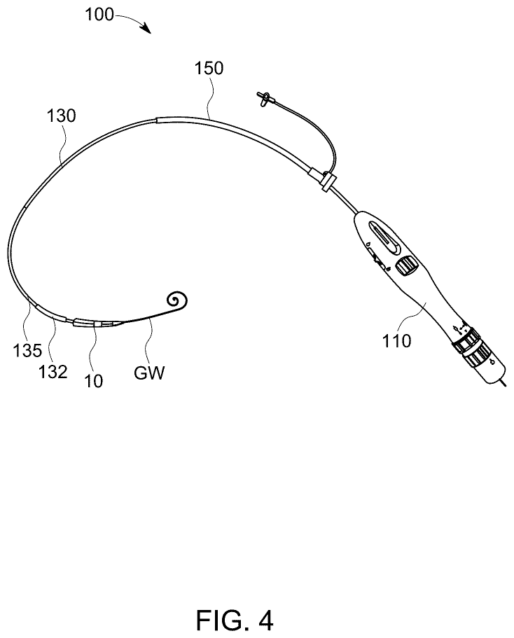

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

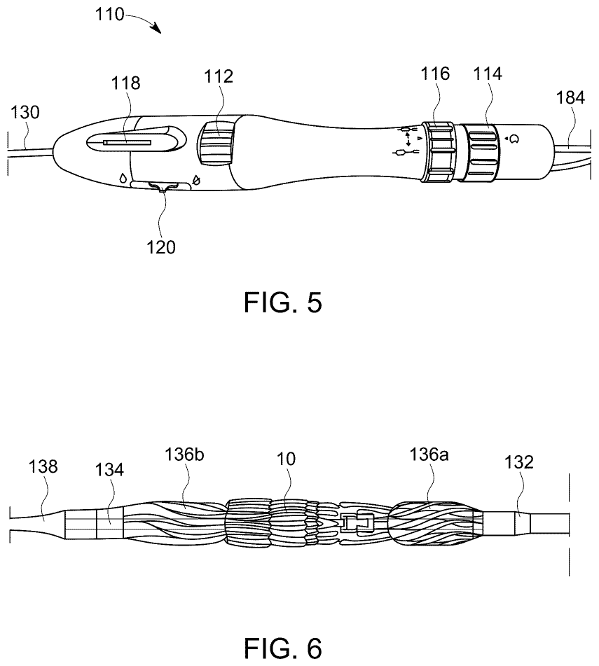

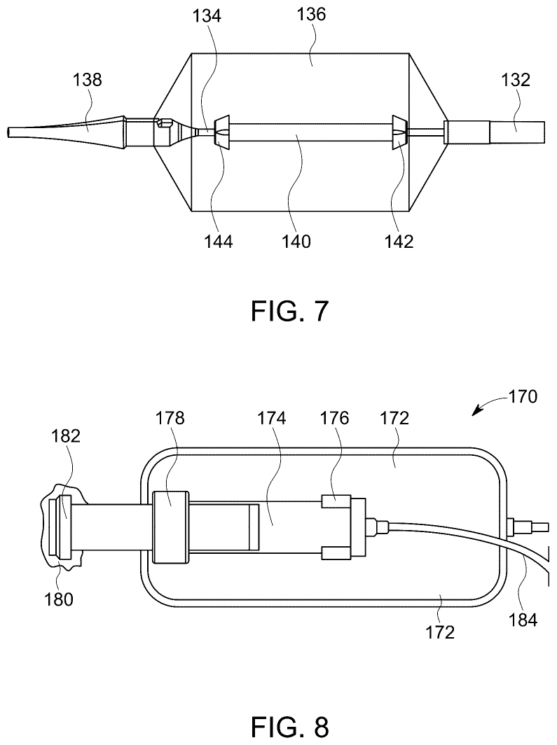

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. Handle 110 may include a slot 118 with an indicator extending therethrough, the indicator moving along the slot 118 as the delivery catheter 130 deflects (e.g., the indicator moves proximally as deflection increases). If included, the indicator and slot 118 may provide the user an easy reference of how much the delivery catheter 130 is deflected at any given point. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads of a carriage that is coupled to the inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Patent Application Publication No. 2024/0148501, the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

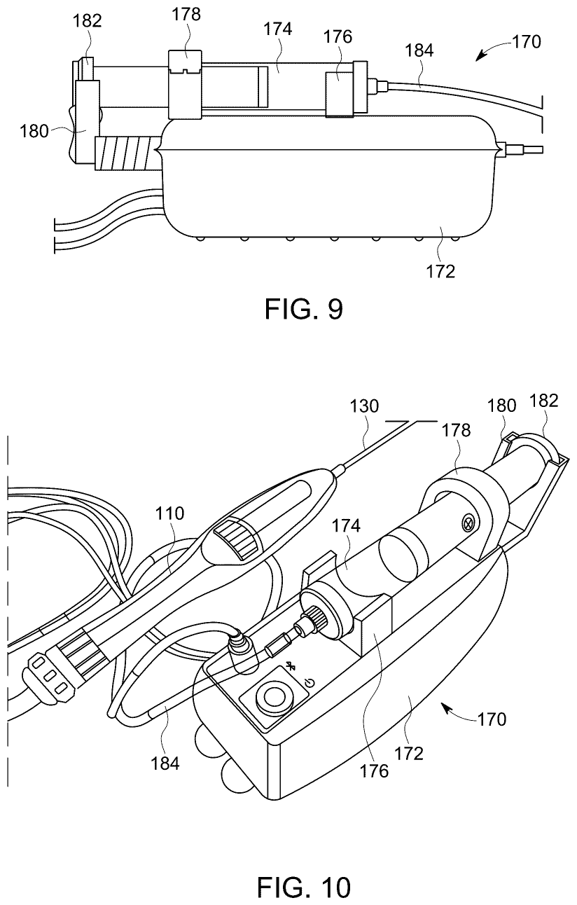

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. As used herein, the phrase “fluid reservoir” and “syringe” may be used interchangeably. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g., from AC mains or another component with which the cable is coupled) and/or transmission of data, information, control commands, etc. For example, one cable may couple the housing 172 to handle 110 so that controls on the handle 110 (e.g., balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. Patent Application Publication No. 2023/0372097, the disclosure of which is hereby incorporated by reference herein.

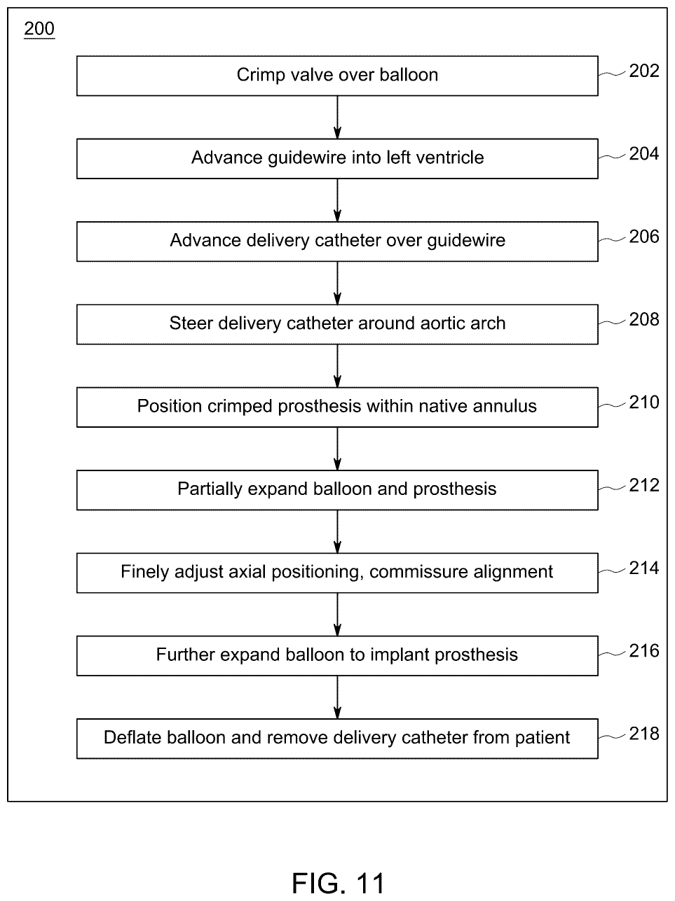

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g., by actuating axial alignment actuator 116) and/or the rotational orientation of the prosthetic heart valve 10 relative to the native aortic valve may be finely adjust (e.g., by actuating commissure alignment actuator 114). When the desired axial alignment is achieve and the desired rotational alignment (e.g., rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel. In one example, the delivery catheter 130 may be guided to the right atrium and/or right ventricle for a tricuspid valve or pulmonary valve procedure. In another example, the delivery catheter 130 may be guided to the left atrium and/or left ventricle for a mitral valve procedure.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

As explained above, transcatheter prosthetic heart valves, including both balloon-expandable and self-expanding prosthetic heart valves, are typically delivered to a patient while in a reduced-diameter collapsed condition. In some examples, it may be important to strike a balance when collapsing a prosthetic heart valve to a smaller size prior to use. For example, if a prosthetic heart valve is not collapsed enough, it may become difficult to deliver in view of the size constraints within patient vasculatures, or it may not be well secured to the delivery device. For example, if a prosthetic heart valve is under-crimped onto a balloon of a delivery device, the prosthetic heart valve may not be sufficiently secured to the balloon during delivery. However, in some examples, if a prosthetic heart valve is collapsed to too small a size (e.g., over-crimped), it can become damaged, for example directly from the forces that cause the prosthetic heart valve to collapse, or otherwise components of the prosthetic heart valve (such as pericardial bovine or porcine tissue forming the prosthetic leaflets) may become prone to increased levels of calcification after implantation.

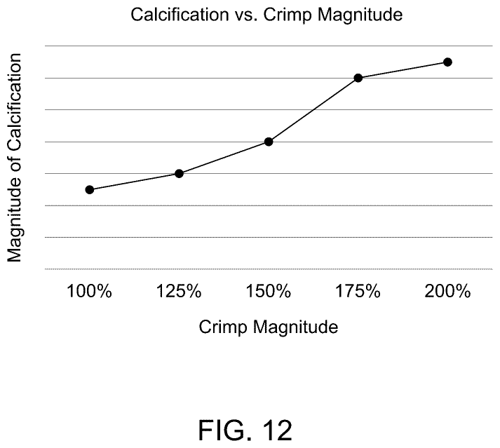

Now referring in addition to FIG. 12, FIG. 12 is a chart that illustrates exemplary data showing the concept of post-implant tissue calcification as a function of crimp magnitude. In the illustrated example of FIG. 12, the x-axis represents crimp magnitude, which in some examples may be calculated as a ratio of the uncompressed volume of the prosthetic heart valve leaflets to the maximum volume of space within the prosthetic heart valve which the prosthetic heart valve leaflets may occupy. Although examples of crimp magnitude are described in greater detail below, in some examples, crimp magnitude may be understood to relate to the extent to which the prosthetic heart valve leaflets have been compressed. In these examples, 100% crimp magnitude may refer to the situation in which the prosthetic heart valve has been crimped to the maximum extent possible prior to the prosthetic heart valve leaflets beginning to meaningfully or demonstrably compress. As the prosthetic heart valve crimps to even smaller sizes (e.g., to smaller diameters or smaller crimp profiles), the prosthetic heart valve leaflets may begin to compress. As crimping continues, the total volume of available space which the prosthetic heart valve which the prosthetic leaflets may occupy decreases, with the uncrimped or uncompressed volume of the prosthetic leaflets remaining constant. Thus, in the example of FIG. 12, 200% crimp magnitude may refer to the situation in which the compressed or crimped volume of the prosthetic leaflets is about half of the uncompressed or uncrimped volume of the prosthetic leaflets. In the example of FIG. 12, the y-axis is an amount of mineralization or calcification that has occurred or is expected to occur after implantation after a given amount of time. Notably, the y-axis is not provided with quantitative values because the graph is meant to illustrate a concept instead of specific data. For example, the example data provided in FIG. 12 illustrate that, all else being equal, a prosthetic heart valve that was crimped to a relatively small diameter and a relatively large amount of compression of the prosthetic leaflets may be expected to have a larger amount of calcification. It should be noted that the data points provided in FIG. 12 are not necessarily representative of specific relationships, but are rather intended to be indicative of general trends.

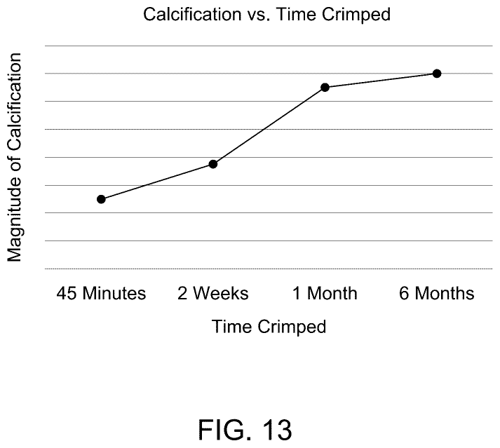

Now referring in addition to FIG. 13, FIG. 13 is a chart that illustrates the concept of post-implant tissue calcification as a function of time that the prosthetic heart valve spent in the crimped condition. In the illustrated example of FIG. 13, the x-axis represents the time that the prosthetic heart valve spent in the crimped condition prior to deployment within a native valve annulus. In one general type of procedure, a prosthetic heart valve may not be crimped or otherwise collapsed to a small diameter until just prior to implantation, such that the prosthetic heart valve may spend relatively little amount of time (e.g., one hour or less, 45 minutes or less, 30 minutes or less, or 15 minutes or less) in the crimped or collapsed condition before being re-expanded into the native heart valve annulus. In another general type of procedure, a prosthetic heart valve is crimped onto a delivery system (see, e.g., FIG. 4) by a manufacturer, and the system may be stored in packaging for an amount of time (e.g., days, weeks, or months) prior to being used as part of prosthetic heart valve implantation. In the example of FIG. 13, similar to the example of FIG. 12, the y-axis is an amount of mineralization or calcification that has occurred or is expected to occur after implantation after a given amount of time. Notably, the y-axis is not provided with quantitative values because the graph is meant to illustrate a concept instead of specific data. For example, the example data provided in FIG. 13 illustrate that, all else being equal, a prosthetic heart valve that was stored in a crimped condition for a relatively large amount of time may be expected to have a larger amount of calcification. It should be noted that the data points provided in FIG. 13 are not necessarily representative of specific relationships, but are rather intended to be indicative of general trends. It should also be understood that the individual factors of crimp magnitude (see FIG. 12) and time crimped (see FIG. 13) can have interactions. For example, when combining a high crimp magnitude with a long crimp duration, a greater magnitude of calcification may be expected to be observed. Further, in some examples, when a high crimp magnitude is applied in areas where there is relatively high stress on the tissue leaflets (e.g. at the commissures), a greater magnitude of calcification may be expected to be observed.

As should be understood for the exemplary data shown in the charts of FIGS. 12-13, the amount of compression of prosthetic heart valve leaflets, as well as the time spent under compression, may be an important factor that should be controlled to optimize performance of the prosthetic heart valve after implantation while also ensuring that the prosthetic heart valve can be reduced to a small enough size that is optimum for transcatheter delivery. Furthermore, if a prosthetic heart valve is provided packaged in a pre-crimped or pre-collapsed configuration by a manufacturer, it may be desirable to closely control the amount of crimping or otherwise the amount of compression experienced by the prosthetic heart valve leaflets so that there is enhanced uniformity among individual products provided to end-users. Even if a prosthetic heart valve is only collapsed to a smaller size by the end-user just prior to use, it may still be desirable to confirm the level of crimping, collapse, and/or tissue compression that occurs while the valve is crimped (including, e.g., during active crimping and/or at equilibrium compaction after active crimping has stopped at valve recoil has occurred). In some instances, variability in crimping may be significantly more likely in situations in which an end user performs the crimping. For example, an end user may use a manual device to crimp the prosthetic heart valve onto a delivery device immediately prior to the prosthetic heart valve being implanted, the manual device relying on a user applying torque on a lever of a crimping device. Even in cases in which the crimper device includes a stop mechanism to limit the amount of crimping possible, high variability may remain in the actual level of crimping and/or compression of prosthetic leaflet tissue. In other words, whether prosthetic heart valve crimping is performed manually immediately prior to use, or by a manufacturer prior to packaging a significant time before use, it may be desirable for various reasons to confirm the amount of crimping that has been performed on the prosthetic heart valve.

In one example, the magnitude of crimping may be quantified by estimating compaction of the prosthetic leaflets. In the particular example below, reference will be made to estimating compaction of prosthetic leaflets (e.g., prosthetic leaflets 90) of a balloon-expandable prosthetic heart valve (e.g., prosthetic heart valve 10). However, it should be understood that the example(s) described below may apply to other balloon-expandable prosthetic heart valves, as well as other collapsible and expandable prosthetic heart valves, with or without modification.

In one example, the compaction may be calculated as a percentage or ratio that compares the pre-crimped or pre-compressed volume of the leaflet tissue (which may be represented by the value Vₜᵢₛₛᵤₑ) to the volume of available space that the leaflet tissue has to occupy when the prosthetic heart valve is at a particular crimp size (which may be represented by the value V_{available}). In this example, the compaction value may be quantified as:

$$Compaction ( \% ) = \frac{{V}_{tissue}}{{V}_{available}} \qquad ( 1 )$$

In some examples, the pre-crimped or pre-compressed volume of the leaflet tissue (Vₜᵢₛₛᵤₑ) may be obtained as a product of the surface area of the prosthetic leaflet (e.g., the area of the surface of prosthetic leaflet 90 shown in FIG. 3), and the thickness of the prosthetic leaflet. In some examples, the surface area may be calculated using computer models (e.g., from computer aided design (“CAD”) drawings). In some examples, the thickness of the prosthetic leaflet may be determined as a maximum thickness. For example, thickness measurements may be taken of the prosthetic leaflet(s) using calipers or other devices, and an average value or the maximum value may be used in calculating the tissue volume. Further, other methods may be suitable for characterizing the thickness (and/or volume) of the pre-crimped leaflet tissue, including microcomputer tomography (microCT), laser scanning, etc.

In some examples, the volume of available space that the leaflet tissue has to occupy when the prosthetic heart valve is at a particular crimp size (e.g., V_{available}) may be determined by prescribing a cylinder (which may also be referred to as a capsule) around the crimped prosthetic heart valve and subtracting the volume of non-tissue components from the volume of the prescribed cylinder. The relevant non-tissue components may depend, in part, on the particular prosthetic heart valve at issue. For example, for self-expandable prosthetic heart valves, the entire valve is within a rigid capsule (e.g., a valve-retaining capsule of a delivery device). In that case, the outer cuff would be considered a non-tissue component that is subtracted from the volume of the prescribed cylinder. However, for balloon expandable prosthetic heart valves, including the one of this example, this outer cuff volume is not included in the subtraction as the frame does not expand on its own. In this example of a balloon-expandable prosthetic heart valve, the volume of available space may be quantified as:

$${V}_{available} = {V}_{capsule} - {V}_{frame} - {V}_{inner cuff} - {V}_{balloon} \qquad ( 2 )$$

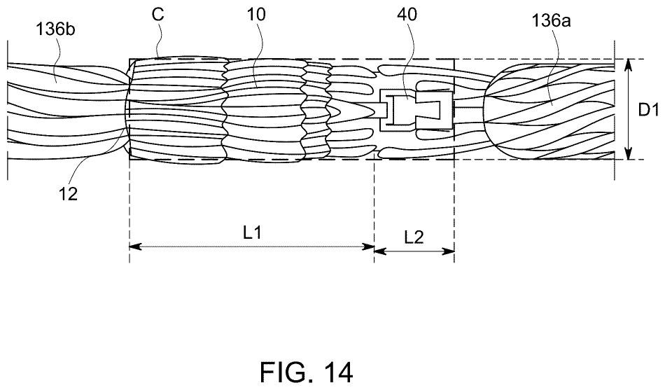

In equation (2) above, the volume of the capsule or cylinder V_{capsule} may be determined in some examples in part by the diameter of the prescribed cylinder (represented by the rectangle in the view of FIG. 14). Now referring in addition to FIG. 14, FIG. 14 illustrates an example of prosthetic heart valve 10 crimped over balloon 136 and positioned between proximal pillow 136a and distal pillow 136b, similar to the example shown in FIG. 6. FIG. 14 shows the cylinder or capsule C outlining the prosthetic heart valve 10, but with the capsule C limited to areas which the prosthetic leaflets 90 may occupy. For example, in the example of FIG. 14, the total length of the capsule may be the sum of the length L1 of the crimped prosthetic heart valve between the inflow end 12 of the prosthetic heart valve 10 and the inflow end of the CAF 40, and the length L2 of the CAF 40 of the prosthetic heart valve 10. Any length of the prosthetic heart valve beyond the outflow end of the CAF 40 may be omitted from the length of the capsule C because the prosthetic leaflets 90 may not be able to occupy that space. In other words, the relevant length may be the length of the prosthetic heart valve which actually contains tissue of the prosthetic leaflets, and the lengths need not be broken down into individual segments as described above. The diameter D1 of the prosthetic heart valve 10 may be calculated as the width of the capsule C (or simply as the diameter of the capsule C). When determining the boundaries of the capsule C, in the specific example of FIG. 14, the outer cuff 80 may be ignored because it is on the outside of the frame 20, and thus does not affect available space which the prosthetic leaflets 90 may occupy. In some examples, the diameter D1 and/or lengths L1, L2 may be determined with a manual measurement (e.g., using a caliper), an image-based measurement (e.g., measuring pixels on a static or live image of the prosthetic heart valve), or any other suitable mode of measurement. In the example of equation (2) above, V_{capsule} may be calculated using the volume of a right cylinder V=πr²h. Referring to the specific example shown in FIG. 14, the volume of the capsule V_{capsule} may be calculated as:

$${V}_{capsule} = {π ( \frac{D 1}{2} )}^{2} ( L 1 + L 2 ) \qquad ( 3 )$$

Referring again to the example equation (2) above, the volume of the stent or frame Vframe may be obtained as a product of the surface area of the frame (e.g., the area of the frame 20, about one-third of which is shown in FIG. 2), and the thickness of the frame. In some examples, the surface area may be calculated using computer models (e.g., from CAD drawings). In some examples, the frame 20 is laser-cut from a tube of material, such as nitinol. In those examples, the thickness of the frame 20 may be determined by the wall thickness of the tube used to form the frame 20. However, in other examples, the thickness of the frame 20 may be determined by the wall thickness of the tube used to form the frame 20, reduced by an average thickness reduction expected from processing steps such as electropolishing, which may tend to reduce the thickness of the frame 20 compared to the original wall thickness of the tube of material. In some examples, thickness measurements may be taken of the frame 20 using calipers or other devices (e.g., before or after processing steps such as electropolishing), and an average value or the maximum value may be used in calculating the volume of the frame V_{frame}. It should be understood that when determining the volume of the frame V_{frame}, structure of the frame extending beyond the capsule C (e.g., structure of the frame 20 beyond the outflow end of the CAFs 40) may be excluded from the calculation. In other words, in some examples, only volume of prosthetic heart valve 10 that may occupy the capsule C is relevant and structure outside of the boundaries of the capsule C may be ignored.

Referring again to the example equation (2) above, the volume of the inner cuff V_{inner cuff} may be obtained as a product of the surface area of the inner cuff (e.g., the area of the inner cuff 60) and the thickness of the inner cuff. In some examples, the surface area may be calculated using computer models (e.g., from CAD drawings of inner cuff 60). In some examples, the thickness of the inner cuff 60 may be determined from a product sheet or specifications (including CAD drawings) of the fabric material from which the inner cuff 60 is cut. In other examples, the thickness of the inner cuff 60 may be determined by taking manual thickness measurements of the inner cuff 60 using calipers or other devices, and an average value or the maximum value may be used in calculating the volume of the inner cuff V_{inner cuff}. It should be understood that when determining the volume of the inner cuff V_{inner cuff}, structure of the inner cuff extending beyond the capsule C (e.g., structure of the inner cuff 60 beyond the outflow end of the CAFs 40) may be excluded from the calculation. In other words, in some examples, only volume of prosthetic heart valve 10 that may occupy the capsule C is relevant and structure outside of the boundaries of the capsule C may be ignored.

Referring again to the example equation (2) above, the volume of the balloon V_{balloon} may be obtained in any suitable fashion. It should be understood that, although there may be components within the balloon (e.g., inner balloon catheter 134 inside of balloon 136), the calculation of the volume of the balloon V_{balloon} encapsulates the volume of any/all components inside the balloon, so those additional components needs not be separately calculated. Further, in some examples, it should be understood that only portions of the balloon that fall within the boundaries of the capsule C need to be accounted for, for similar reasons as described above in connection with the frame 20 and the inner cuff 60. In one particular example, this crimp zone is the portion of the balloon between the pillows (e.g., the portion of balloon 136 between proximal pillow 136a and distal pillow 136b, which may be referred to as the “crimp zone” of the balloon 136). In some examples, the crimp zone may be assumed to be a right cylinder. In these examples, the volume of the balloon V_{balloon} may be calculated by calculating the volume of a right cylinder, similar to that described above, where the length or height of the cylinder is limited to the lengths L1 and L2 of the compartment C, with the radius being half the diameter, for example as explained above.

In some examples of balloon-expandable prosthetic heart valves, such as prosthetic heart valve 10, the information provided above may allow for a complete calculation of the crimp magnitude of compaction percentage of equation (1) above. In some examples, when crimping a prosthetic heart valve, the diameter of the prosthetic heart valve reaches a minimum value when a maximum crimping force is applied (which may be referred to as “maximum compaction”), but after the crimping force is released, recoil may occur in which the diameter of the prosthetic heart valve slightly increases (which may be referred to as “equilibrium compaction”). The equations provided above (or similar equations) may be used to calculate compaction ratios for both the maximum compaction and the equilibrium compaction. However, because the prosthetic heart valve is typically only in the state of maximum compaction for a relatively small amount of time, with the remainder of time after crimping (and before implantation) being spent in the state of equilibrium compaction, the equilibrium compaction value may have a higher relevance (although, as is explained below, the maximum compaction value may also be valuable to use). However, for self-expandable prosthetic heart valves, because the valve is contained within a rigid capsule that controls the diameter of the collapsed valve (without the valve having any recoil), the maximum compaction and the equilibrium compaction may be the same. It should be understood that, in some examples, the various calculations provided above may reflect bulk volumes, and localized compaction may vary slightly based on how the actual different components collapse during the crimping process. Nonetheless, the bulk volume calculations may provide useful information despite potential local variances.

The example(s) above relied on volumes (or estimated volumes) of components of the prosthetic heart valve to determine compaction values (or crimp magnitudes). In other examples, instead of using a three-dimensional approach, a two-dimensional approach may be used. In such examples, the compaction may be calculated as a percentage or ratio that compares the pre-crimped or pre-compressed cross-sectional area of the leaflet tissue (which may be represented by the value Aₜᵢₛₛᵤₑ) to the cross-sectional area of available space that the leaflet tissue has to occupy when the prosthetic heart valve is at a particular crimp size (which may be represented by the value A_{available}). In these examples, the compaction value may be quantified as:

$$Compaction ( \% ) = \frac{{A}_{tissue}}{{A}_{available}} \qquad ( 4 )$$

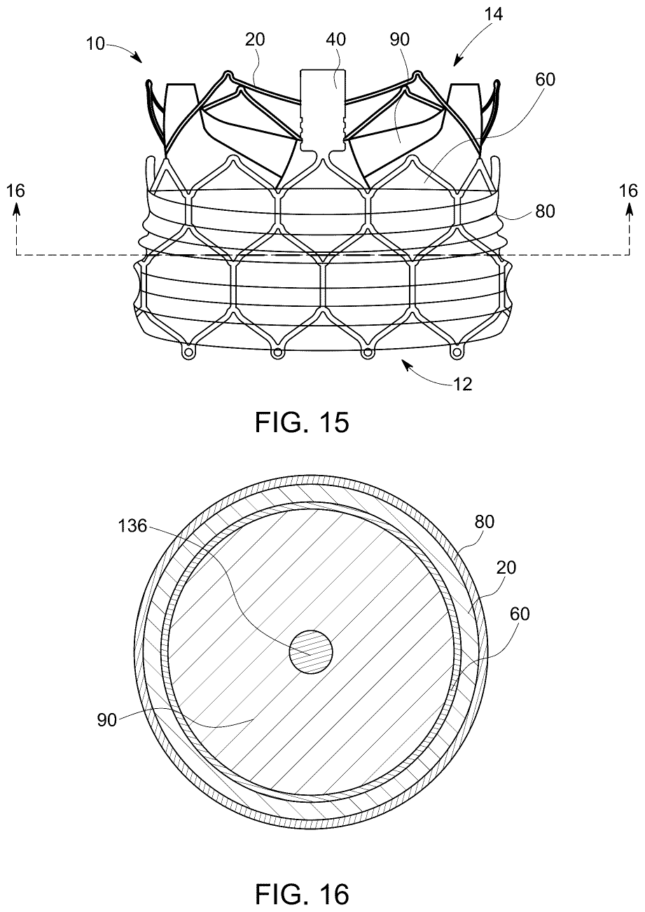

In some examples, the pre-crimped or pre-compressed area of the leaflet tissue (Aₜᵢₛₛᵤₑ) may be determined as the cross-sectional area of the prosthetic leaflets (e.g., the cross-sectional area of an assembly of prosthetic leaflets 90 shown in FIG. 3). For example, referring now in addition to FIG. 15, FIG. 15 is a side view of prosthetic heart valve 10 illustrating a section line 16-16. Section line 16-16 may be taken along an axial length of the prosthetic heart valve 10 expected to have the greatest packing of material of the prosthetic heart valve 10. In the particular illustrated example, section line 16-16 may be taken orthogonal to the central longitudinal axis of the prosthetic heart valve 10 at an axial midpoint between the inflow end 12 of the prosthetic heart valve and an inflow end of the CAF 40 of the prosthetic heart valve 10. However, this axial midpoint is only one example of where the cross-section may be taken, and other examples may be suitable. An example of the cross-section of prosthetic heart valve 10, after being mounted to a delivery device, taken along section line 16-16 is shown in FIG. 16. Referring now in addition to FIG. 16, the stack-up of components of prosthetic heart valve 10, when crimped on a balloon (e.g., balloon 136) of a delivery system (e.g., delivery system 100), from the radial outermost component to the radial innermost component, includes the outer cuff 80, frame 20, inner cuff 60, prosthetic leaflets 90, and balloon 136 (which may encompass components within the balloon, such an inner balloon catheter such as inner balloon catheter 134 which is omitted from FIG. 16 for clarity of illustration).

In some examples, the pre-crimped or pre-compressed cross-sectional area of the leaflet tissue (Aₜᵢₛₛᵤₑ) may be obtained in substantially the same way as described above for determining the pre-crimped or pre-compressed volume of the leaflet tissue (Vₜᵢₛₛᵤₑ), except that length is not accounted for. For example, prior to the tissue of the prosthetic leaflets 90 beginning to compress, an annular area of the prosthetic leaflets 90 may be calculated, for example using the equation:

$${A}_{tissue} = π ( {R}^{2} - {r}^{2} ) \qquad ( 5 )$$

In the example of equation (5) above, the radius R value may be the radius between the radial center of the prosthetic heart valve (e.g., the center of FIG. 16) and the outer surface of the prosthetic leaflets, while the radius r value may be the radius between the radial center of the prosthetic heart valve and the inner surface of the prosthetic leaflets, while the assembly of the prosthetic leaflets 90 has a substantially circular configuration. It should be understood that, in this example, the difference between radius R and radius r may represent the thickness of the leaflets 90. However, in other examples, the area may be calculated in a different way. For example, the thickness of the various components of the system may be estimated (e.g. by measuring it on the bench at the component level before assembling the prosthetic heart valve together) to estimate the relevant areas. It should be understood that this option for estimating area may be performed for components in addition to just the prosthetic leaflets.

In some examples, the cross-sectional area of available space that the leaflet tissue has to occupy when the prosthetic heart valve is at a particular crimp size (e.g., A_{available}) may be determined by prescribing a circle around the outer surface of the frame, and subtracting the cross-sectional of non-tissue components from the cross-sectional area of the prescribed circle. In this example the volume of available space may be quantified as:

$${A}_{available} = {A}_{circle} - {A}_{frame} - {A}_{inner cuff} - {A}_{balloon} \qquad ( 6 )$$

In equation (6) above, the cross-sectional area the circle A_{circle} may be determined in some examples by projecting a circle over outside of the crimped frame of the prosthetic heart valve, such as a circle around the outer surface of frame 20 in FIG. 16. It should be understood, as explained above, that equation (6) may be used for the example of a balloon-expandable prosthetic heart valve, but other components (such as an outer cuff) may be included in the equation for other prosthetic heart valves (e.g. for self-expanding prosthetic heart valves). The area of A_{circle} may be calculated using the formula for the area of circle, for example:

$${A}_{circle} = π {r}^{2} \qquad ( 7 )$$

In equation (7), the radius r may be the radius of the projected circle. Referring again to the example equation (6) above, the area of the stent or frame and the area of the inner cuff in some examples may be calculated using substantially the same calculation for the area of the tissue explained in connection with equation (5), where the frame and the inner cuff are treated as having an annular shape. The area of the balloon A_{balloon} in some examples may be calculated in the same way as described above for the volume of the balloon V_{balloon}, except that the length component is ignored, allowing the area of the balloon A_{balloon} to be calculated as the area of a circle having a radius that is the radius of the balloon (e.g., the radius of the crimp zone of balloon 136).

In some examples of balloon-expandable prosthetic heart valves, such as prosthetic heart valve 10, the information provided above may allow for a complete calculation of the crimp magnitude of compaction percentage of equation (4) above. As with the calculation of the crimp magnitude using volumes, calculating a crimp magnitude using areas may be performed at either or both of the state of maximum compaction as well as the state of equilibrium compaction, for substantially the same reasons as described above.

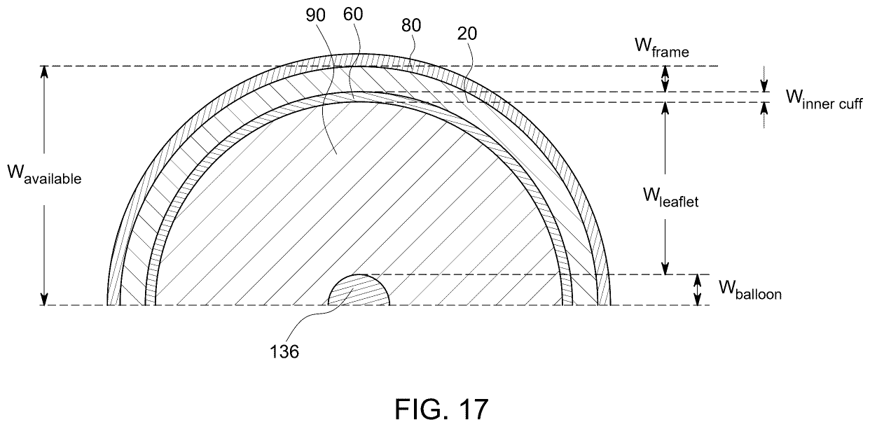

The example(s) above relied on volumes (or estimated volumes) or cross-sectional areas (or estimated cross-sectional areas) of components of the prosthetic heart valve to determine compaction values (or crimp magnitudes). In yet another example, instead of using a two- or three-dimensional approach, a one-dimensional approach may be used. In such examples, the compaction may be calculated as a percentage or ratio that compares the pre-crimped or pre-compressed thickness (or width) of the leaflet tissue to the thickness (or width) of available space that the leaflet tissue has to occupy when the prosthetic heart valve is at a particular crimp size. For example, referring now in addition to FIG. 17, the calculations may be the same as provided for the volume and area methods described above, but use only a single thickness or width dimension. In other words, in some examples, the total available space W_{available} may be calculated as the radius between the center of the balloon 136 and the outer surface of the frame 20. The thickness or width of the leaflet W_{leaflet} may be calculated as the thickness (e.g., average or maximum measured thickness) of the leaflet 90 before it has begun to compress. The thickness or width of the frame W_{frame} may be calculated as the wall thickness of the frame 20, either before or after processing. For example, as described above, the thickness or width of the frame W_{frame} may account for thickness or width reduction that results from electropolishing. In some examples, the thickness or width of the frame W_{frame} may be calculated as an average of measurements or a maximum width or thickness measurement. The thickness or width of the inner cuff W_{inner cuff} may be relatively straightforward measurement of the thickness of the inner cuff 60 (including either an average of measurements or a maximum measurement), which may be manually measured or taken from a spec sheet for the material (e.g., fabric) used to form the inner cuff 60. The calculations may otherwise be performed in substantially the same way as described above for the volume and area examples, where the crimp magnitude is determined based on the ratio of the uncompressed thickness of the leaflet W_{leaflet} compared to the total amount of width or thickness available W_{available} for the leaflet 90 to occupy.

In each of the examples above, whether calculating crimp magnitude using three-, two-, or one-dimensional methods, the result of the calculation is a ratio or percentage that indicates a level of compression of the tissue-based prosthetic leaflets 90. For example, when the prosthetic heart valve 10 is crimped to the maximum possible amount it can be crimped before causing the prosthetic leaflets to begin to compress, the crimp magnitude is about 1 or 100%. As the size of the prosthetic heart valve 10 continues to reduce (e.g., by further crimping), the other components of the prosthetic heart valve 10 (including the balloon 136 over which the prosthetic heart valve 10 is being crimped) may not undergo compression of the material, or at least may not undergo nearly as significant an amount of material compression compared to the tissue leaflets. This may be because, for example, the tissue leaflets 90 store water (and/or other fluids) within the structure of the leaflets 90, allowing the leaflets 90 to compress in size as water (and/or other fluids) are squeezed out of the leaflets 90. Thus, a crimp magnitude of 2 or 200% may indicate that the prosthetic leaflets 90 are occupying only half the space that the prosthetic leaflets 90 occupied prior to being compressed, and so forth. As another example, if the size of the prosthetic tissue leaflets in an uncompressed condition is 10 units of volume, and the amount of available size that the prosthetic tissue leaflets can occupy when the prosthetic tissue leaflets are crimped or compressed is 8 units of volume, the ratio is 10:8 or 1.25 or 125% compaction. As should be clear, the crimp magnitude may provide a useful and largely objective calculation of the extent to which the prosthetic leaflets 90 have been compressed as a result of collapsing (e.g., crimping or otherwise reducing the size if the prosthetic heart valve). Notably, this calculation may be performed on a valve-by-valve basis. In other words, even if manufacturing tolerances are very tight and the frame 20, inner cuff 60, and balloon 136 can be produced with very little variation in sizing (for a given size of the prosthetic heart valve), tissue leaflets 90 are typically prone to significantly more variation in sizing given their biological basis. In other words, even if two otherwise identical size 29 mm prosthetic heart valves are manufactured, differences in thickness of the tissue leaflets for each prosthetic heart valve may mean that, even if the two prosthetic heart valves are crimped to the same exact outer diameter, the tissue leaflets may compress different amounts, resulting in different crimp magnitudes. Stated in another way, even if a manual crimping device is provided with a stop mechanism to attempt to ensure that all prosthetic heart valves crimped with the manual crimping device are crimped to the same diameter, there may still be significant variation in the amount of compression that occurs within the tissue leaflets of each prosthetic heart valve. The same may be true if crimping of the prosthetic heart valves is done by a more precise machine used by the manufacturer when providing a prosthetic heart valve in a pre-crimped or pre-mounted configuration. Thus, the methods of determining a crimp magnitude for a prosthetic heart valve may be applied in various ways to achieve benefits, particular examples of which are provided in more detail below.

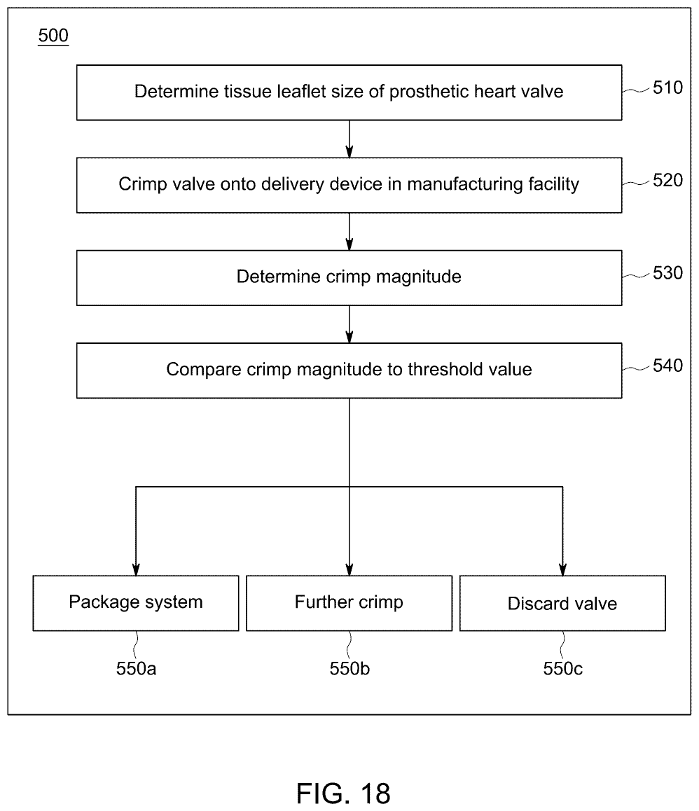

Referring now in addition to FIG. 18, FIG. 18 is a flow chart showing example steps of an example method 500 of using crimp magnitude in a manufacturing process. This particular example relates to a situation in which a prosthetic heart valve (e.g., prosthetic heart valve 10) is being collapsed (e.g., crimped) by the manufacturer so that the prosthetic heart valve is packaged and stored in the collapsed condition, for example crimped over a balloon of a delivery device. In some examples, method 500 may include a step 510 in which the size of the uncompressed tissue leaflets of a particular prosthetic heart valve being collapsed for storage and/or packaging is determined. In some examples, the step 510 may be performed using a volumetric measurement method (e.g., three-dimensional), an area measurement method (e.g., two-dimensional), or a thickness measurement method (e.g., one-dimensional). In some examples, after completing step 510, the prosthetic heart valve is transitioned to a collapsed condition within or on a delivery device in step 520, which may be performed prior to packaging the system. After the collapsing or crimping step 520, in some examples the crimp magnitude may be determined in step 530. In some examples, the crimp magnitude may be determined by any of the methods described above (e.g., one-, two-, or three-dimensional calculations). Step 530 may in some examples be performed using a different specific calculation than those described above, as long as the calculated crimp magnitude reflects an amount of compression of the prosthetic tissue leaflets. In some examples, step 530 may be performed more than once, including for example to determine both a maximum compaction and an equilibrium compaction. After determining the crimp magnitude in step 530, in some examples, the determined crimp magnitude may be compared to a threshold value of crimp magnitude. In some examples, the threshold value may be a crimp magnitude which has been determined to provide an acceptable risk of calcification of the tissue leaflets after implantation. For example, referring back to FIG. 12, it may have been determined that a crimp magnitude of 150% is the maximum threshold for crimp magnitude which should be used in manufacturing to provide an appropriately small risk of calcification. It is worth reiterating here that the specific data shown in FIG. 12 is merely exemplary and intended to illustrate trends as opposed to specific data, and the 150% crimp magnitude is only an exemplary threshold any other may be used. Notably, referring briefly back to FIG. 13, the threshold crimp magnitude may depend on an expected time that the prosthetic heart valve is expected to remain in the crimped condition before use. So, as an example, if it is known that the prosthetic heart valve will remain crimped in storage and/or packaging for less than two weeks, the crimp magnitude threshold may be set to a higher amount than an identical system for which it is expected that the prosthetic heart valve will remain crimped in storage and/or packaging for two months. It should be understood that, in some examples, step 540 may be performed twice to compare a maximum crimp magnitude determined in step 530 to a threshold value for acceptable maximum crimp magnitude, and also to compare an equilibrium crimp magnitude determined in step 530 to a threshold value for acceptable equilibrium crimp magnitude.

Referring again to FIG. 18, the crimp magnitude determined in step 530 may be compared to the threshold crimp magnitude value in step 540. In some examples, the threshold crimp magnitude may be treated as an absolute maximum. In other examples, the threshold crimp magnitude may be treated with an allowable deviation. For example, if the threshold crimp magnitude is set as 150%, any value of crimp magnitude determined in step 530 to be greater than 150% may be considered unacceptably high. In other examples, if the threshold crimp magnitude is set as 150%, any value of crimp magnitude determined in step 530 to be greater than 150% by more than a tolerance value (e.g., a 5% tolerance value resulting a true cut-off of 155%) may be considered unacceptably high. If the crimp magnitude determined in step 530 is determined to be unacceptably high in step 540, the prosthetic heart valve may be considered compromised and discarded in step 550c. If the crimp magnitude determined in step 530 is not determined to be unacceptably high in step 540, the manufacturing and/or assembly process may continue and the system may be packaged for sale and/or distribution in step 550a. In some examples, having a crimp magnitude that is smaller than the threshold value is not considered to create any safety risk, but it may nonetheless be desirable to achieve a crimp magnitude closer to the threshold value to provide a smaller crimp profile of the prosthetic heart valve within the determined acceptable range of crimp magnitudes. In those instances, the prosthetic heart valve may be further crimped in step 550b, at which point the method steps 530 and 540 may be performed again to assess the resulting crimp magnitude. It should be understood that method 500 may include additional or alternative steps not shown in FIG. 18. However, as should be clear from the above description, the method 500 of FIG. 18 and similar methods may allow for a highly consistent manufacturing process in which every prosthetic heart valve that is pre-mounted or pre-crimped to a delivery system is crimped or otherwise collapsed to a desirable size, even considering variations between the tissue leaflets between different prosthetic heart valves.

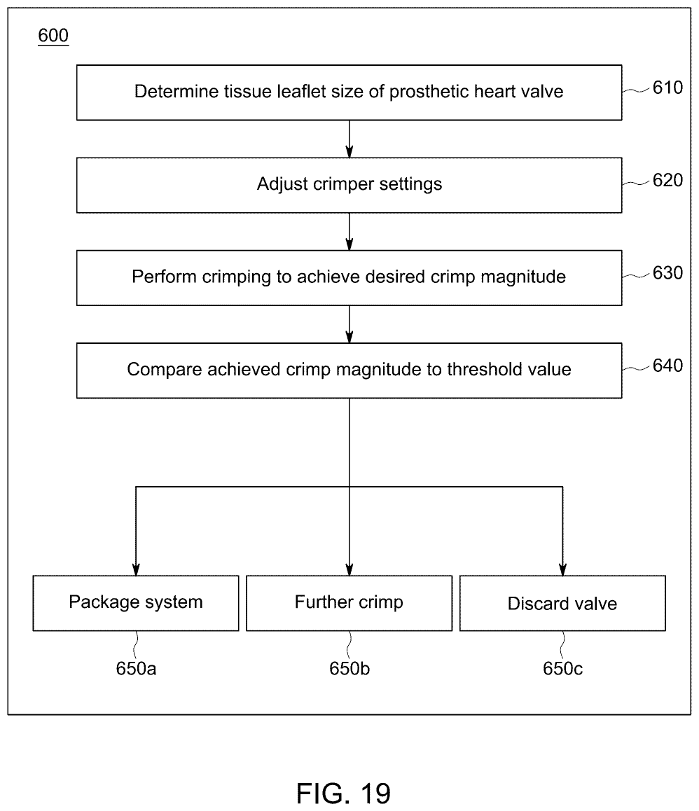

Referring now in addition to FIG. 19, FIG. 19 is a flow chart showing example steps of an example method 600 of using crimp magnitude in a manufacturing process. Method 600 has items in common with method 500. For example, the particular example of method 600 also relates to the situation in which a prosthetic heart valve (e.g., prosthetic heart valve 10) is being collapsed (e.g., crimped) by the manufacturer so that the prosthetic heart valve is packaged and stored in the collapsed condition, for example crimped over a balloon of a delivery device. In some examples, method 600 may include a step 610 in which the size of the uncompressed tissue leaflets of a particular prosthetic heart valve being collapsed for storage and/or packaging is determined. In some examples, the step 610 may be performed using a volumetric measurement method (e.g., three-dimensional), an area measurement method (e.g., two-dimensional), or a thickness measurement method (e.g., one-dimensional). In some examples, after completing step 610, it may be determined what level of crimping (e.g., how much the prosthetic heart valve should be collapsed) is necessary to achieve a crimp magnitude that is at, under, or within a safe tolerance range of a threshold crimp magnitude. The threshold crimp magnitude in some examples may be the same as that described in connection with method 500, and is thus not described in detail again here. For example, if a prosthetic heart valve is determined to have relatively thin tissue leaflets (when the tissue leaflets are uncompressed), it may be determined that the prosthetic heart valve can be safely crimped to a relatively small diameter. However, if a prosthetic heart valve is determined to have relatively thick tissue leaflets (when the tissue leaflets are uncompressed), it may be determined that the prosthetic heart valve should be crimped to a relatively large diameter to maintain a safe crimp magnitude. Thus, in some examples, in step 620 the crimper (which may be an automated, motorized crimper) may be set to crimp the prosthetic heart valve to a target diameter that achieves a desired crimp magnitude. In some examples, in step 630, crimping is performed using the adjusted crimp settings from step 620, resulting in the prosthetic heart valve being crimped to a particular size in an attempt to achieve the desired crimp magnitude. In some examples, despite the crimper being set to a particular crimp value for the tissue leaflets of that prosthetic heart valve, it may be desirable to perform the same quality check as described in connection with method 500. For example, in some examples step 640 may be performed to compare the achieved crimp magnitude to the threshold value, and in some examples, depending on the result, the prosthetic heart valve may be packaged for sale and/or distribution in step 650a, further crimping may be performed in step 650b, or the prosthetic heart valve may be discarded in step 650c. Because steps 640 and 650a-650c are substantially the same as their counterpart steps 540 and 550a-550c in method 500, they are not described in further detail again here.

Although exemplary methods 500 and 600 may be particularly useful in situations in which manufacturing is being performed with an automated and/or motorized crimper with high precision to provide a prosthetic heart valve that is pre-mounted or pre-crimped to a delivery system, it should be understood that similar methods using similar concepts may be incorporated into more traditional situations in which crimping is being performed manually by an end-user immediately prior to implantation of the prosthetic heart valve.

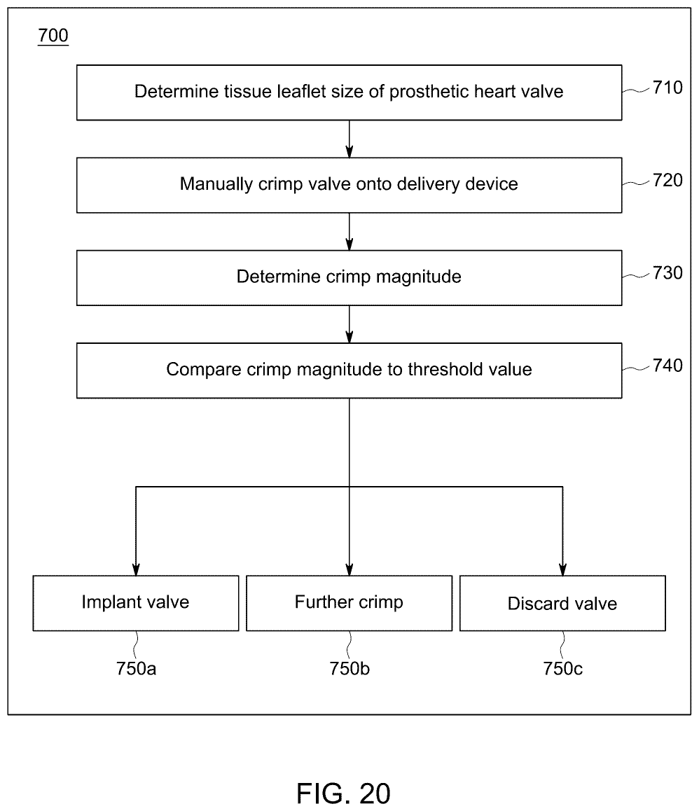

For example, referring now in addition to FIG. 20, FIG. 20 is a flow chart showing example steps of an example method 700 of using crimp magnitude as part of an implantation procedure. Method 700 has items in common with methods 500, 600, but focuses on the use of crimp magnitude when the prosthetic heart valve is being crimped just prior to implantation, for example by an end user in a hospital using a manual crimper (although it should be understood that method 700 may apply to using an automated crimper). In some examples, method 700 may include a step 710 in which the size of the uncompressed tissue leaflets of a particular prosthetic heart valve being implanted is determined. In some examples, step 710 may be performed by the end user, although in other examples, step 710 may be performed during an earlier manufacturing or assembly step, and the information determined may be provided to the end user along with the prosthetic heart valve. In some examples, the step 710 may be performed using a volumetric measurement method (e.g., three-dimensional), an area measurement method (e.g., two-dimensional), or a thickness measurement method (e.g., one-dimensional). In some examples, after completing step 710, the prosthetic heart valve is transitioned to a collapsed condition within or on a delivery device in step 720. In some examples, step 720 is performed as part of the overall implantation procedure by the end user or personnel associated with the end user. In some examples, step 720 may be performed using a traditional manual crimper or using an automated and/or motorized crimper. In some examples, step 720 is performed by inserting the prosthetic heart valve into the crimper while the prosthetic heart valve has an initial diameter in which the prosthetic tissue leaflets are uncompressed, and then the prosthetic heart valve is crimped to a smaller crimped diameter in which the prosthetic tissue leaflets are compressed. After the collapsing or crimping step 720, in some examples the crimp magnitude may be determined in step 730. The crimp magnitude in step 730 may be performed in substantially the same way as described in connection with step 530 of method 500, and is thus not described again here. After determining the crimp magnitude in step 730, in some examples, the determined crimp magnitude may be compared to a threshold value of crimp magnitude in step 740. The crimp magnitude comparison in step 740 may be performed in substantially the same way as described in connection with step 540 of method 500, and is thus not described again here. Based on the results of step 740, up to three or more different actions may be taken. For example, if the comparison of step 740 results in an acceptable crimp magnitude compared to threshold crimp magnitude, the procedure may continue and the prosthetic heart valve may be implanted in step 750a. Step 750a is the corollary to steps 550a, 650a, but because the prosthetic heart valve is intended for immediate use in method 700, instead of packaging the prosthetic heart valve, it is used. However, in some examples, the prosthetic heart valve is only in the crimped condition for a relatively short amount of time between crimping and implantation/deployment, for example one hour or less, 45 minutes or less, 30 minutes or less, or 15 minutes or less. Otherwise, if the comparison step 740 results in an unacceptably high crimp magnitude compared to threshold value, the valve may be discarded in step 750c. In some examples, if the comparison step 740 indicates that the prosthetic heart valve may be safely further crimped, an additional crimping step 750b may be performed, and then method may return to step 730 in such an example. Because steps 750b and 750c are substantially similar to corollary steps 550b, 650b and 550c, 650c, they are not described in greater detail here. It should be understood that, because method 700 contemplates immediate use of the prosthetic heart valve, and thus a shorter time spent in the crimped condition compared to methods 500, 600, the threshold value for crimp magnitude may be set to a higher value in method 700 compared to methods 500, 600, at least in some examples. Further, as with methods 500, 600, certain steps may be omitted, added, or changed within method 700.

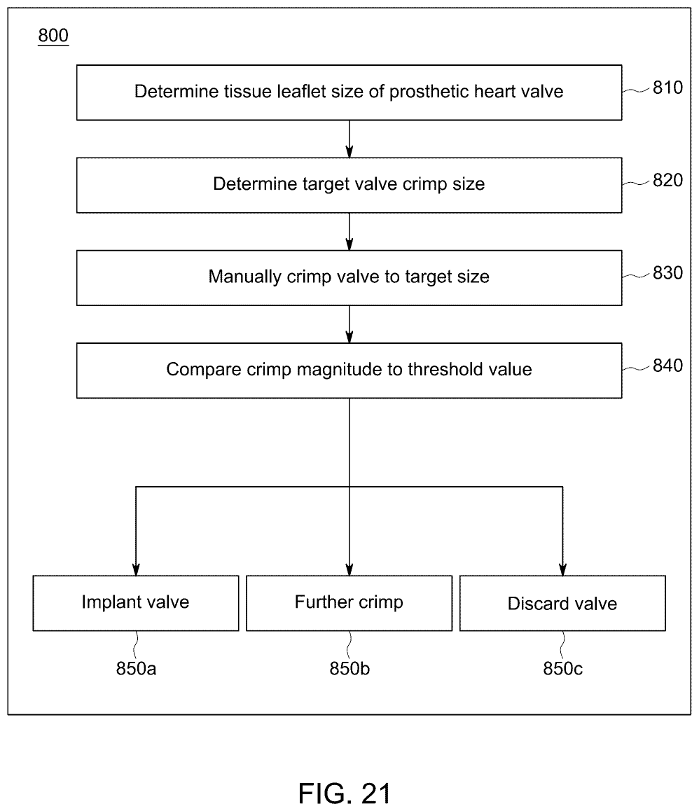

Referring now in addition to FIG. 21, FIG. 21 is a flow chart showing example steps of an example method 800 of using crimp magnitude as part of an implantation procedure. Method 800 has items in common with method 700. For example, the particular example of method 800 also relates to the situation in which a prosthetic heart valve (e.g., prosthetic heart valve 10) is being collapsed (e.g., crimped) by the end user (or someone associated with the end user) for immediate use in an implantation procedure. In some examples, method 800 may include a step 810 in which the size of the uncompressed tissue leaflets of a particular prosthetic heart valve being collapsed for immediate implantation is determined. As with step 710, step 810 may be performed by the end user, someone associated with the end user, or during an earlier manufacturing and/or assembly process, with the information being provided to the end user along with the prosthetic heart valve. In some examples, step 810 may be performed using a volumetric measurement method (e.g., three-dimensional), an area measurement method (e.g., two-dimensional), or a thickness measurement method (e.g., one-dimensional). In some examples, after completing step 810, it may be determined what level of crimping (e.g., how much the prosthetic heart valve should be collapsed) is necessary to achieve a crimp magnitude that is at, under, or within a safe tolerance range of a threshold crimp magnitude. The threshold crimp magnitude in some examples may be the same as that described in connection with methods 500, 700, and is thus not described in detail again here. For example, if a prosthetic heart valve is determined to have relatively thin tissue leaflets (when the tissue leaflets are uncompressed), it may be determined that the prosthetic heart valve can be safely crimped to a relatively small diameter. However, if a prosthetic heart valve is determined to have relatively thick tissue leaflets (when the tissue leaflets are uncompressed), it may be determined that the prosthetic heart valve should be crimped to a relatively large diameter to maintain a safe crimp magnitude. Thus, in some examples, in step 820 a target crimp size for the prosthetic heart valve is selected. In some examples, in step 830, crimping is performed (manually or using an automated or motorized crimper) using the target crimp size from step 820, resulting in the prosthetic heart valve being crimped to a particular size in an attempt to achieve the desired crimp magnitude. In some examples, despite the use of a target crimp size, it may be desirable to perform the same quality check as described in connection with method 700. For example, in some examples step 840 may be performed to compare the achieved crimp magnitude to the threshold value, and in some examples, depending on the result, the prosthetic heart valve may be implanted in step 850a, further crimping may be performed in step 850b, or the prosthetic heart valve may be discarded in step 850c. Because steps 840 and 850a-850c are substantially the same as their counterpart steps 740 and 750a-750c in method 700, they are not described in further detail again here. As with methods 500, 600, 700, it should be understood the certain steps may be omitted from, added to, or swapped with other steps in method 800. For example, step 800 may be performed such that step 840 is omitted, and after performing step 830, the valve is immediately implanted in step 850a without further comparisons or checks. It should be understood that, in typical implantation procedures of balloon-expandable prosthetic heart valves today, manual crimping is typically performed immediately prior to the use of prosthetic heart valve without any type of confirmation of crimp magnitude or any other checks on the suitability of the resulting crimp profile.

For the various embodiments described above, the threshold value of crimp magnitude may be different than the specific examples provided. For example, although an example of 150% is used in examples above, in other examples, the threshold crimp magnitude may be anywhere between about 110% and about 200%, particularly in situations in which the prosthetic heart valve is going to be stored for a duration in packaging, as opposed to being implanted very shortly after crimping. In other examples, including those in which the crimping is being performed just prior to implantation, the threshold crimp magnitude may be anywhere between about 110% and about 300%. In other words, a larger crimp magnitude may be suitable in situations in which the prosthetic heart valve is only crimped for a short duration before implantation. In some examples, for balloon-expandable valves, the threshold value of crimp magnitude is the equilibrium compaction value, as opposed to the maximum compaction value (e.g., after crimping force has been removed and some amount of recoil may have occurred), and the threshold crimp magnitude is between about 110% and about 135%. For example, referring briefly to FIGS. 12-13, a crimp magnitude of between about 110% and about 135% may provide a small enough prosthetic heart valve for transcatheter delivery, without creating too high risk of calcification, which may be true even for the longer time periods of crimp shown in FIG. 13. In some examples, for self-expandable valves, the threshold value of crimp magnitude (which may be either the equilibrium compaction value or the maximum compaction value as those parameters are the same for self-expanding valves) is between about 95% and about 125%.

Although various examples of methods are described above to determine a crimp magnitude that relates to compression of tissue leaflets, it should be understood that the methods may be modified or adapted to prosthetic heart valves that are different than the examples provided herein. For example, for a prosthetic heart valve that is identical to prosthetic heart valve 10 but that omits an inner cuff 60, the methodology may substantially the same as described above except that values for an inner cuff are omitted from the crimp magnitude determination. Further, while the main example described herein is for a balloon-expandable prosthetic heart valve, the same concepts may be used in connection with a self-expandable prosthetic heart valve. However, for self-expandable prosthetic heart valves, because the crimp magnitude is effectively determined externally by the size of the delivery system (e.g., the size of the capsule), the concepts described herein may be used to determine a desired size of a delivery device capsule to achieve the desired level of crimp magnitude.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of implanting a prosthetic heart valve, the method comprising:
inserting the prosthetic heart valve into a crimper, the prosthetic heart valve including a frame, an inner cuff disposed on a luminal surface of the frame, and a plurality of prosthetic tissue leaflets mounted within the frame, the prosthetic heart valve having an initial diameter when inserted into the crimper;
while the prosthetic heart valve is inserted into the crimper, crimping the prosthetic heart valve onto a delivery device so that the prosthetic heart valve has a crimped diameter that is smaller than the initial diameter, wherein when the prosthetic heart valve has the initial diameter, the prosthetic tissue leaflets are in an uncompressed condition, and when the prosthetic heart valve has the crimped diameter, the prosthetic tissue leaflets are in a compressed condition;
advancing the prosthetic heart valve into a vasculature of a patient while the prosthetic heart valve has the crimped diameter; and
deploying the prosthetic heart valve into a valve annulus of the patient;
wherein after crimping the prosthetic heart valve from the initial diameter to the compressed diameter, the prosthetic heart valve has a crimp magnitude of between 110% and 135%, the crimp magnitude being calculated as a ratio of (i) a size of the prosthetic tissue leaflets in the uncompressed condition to (ii) a total amount of available size that the prosthetic tissue leaflets can occupy when the prosthetic tissue leaflets are in the compressed condition.

2. The method of claim 1, wherein the size of the prosthetic tissue leaflets is a volume of the prosthetic tissue leaflets, and the total amount of available size that the prosthetic tissue leaflets can occupy is a total amount of available volume that the prosthetic tissue leaflets can occupy.

3. The method of claim 2, wherein the total amount of available volume that the prosthetic tissue leaflets can occupy is calculated as a volume of a cylinder prescribed around an outer surface of the frame, less a volume of the frame within the cylinder, less a volume of the inner cuff within the cylinder, less a volume of delivery device components within the cylinder.

4. The method of claim 1, wherein the size of the prosthetic tissue leaflets is a cross-sectional area of the prosthetic tissue leaflets, and the total amount of available size that the prosthetic tissue leaflets can occupy is a total amount of available cross-sectional area that the prosthetic tissue leaflets can occupy.

5. The method of claim 1, wherein the size of the prosthetic tissue leaflets is a thickness of the prosthetic tissue leaflets, and the total amount of available size that the prosthetic tissue leaflets can occupy is a total amount of available thickness the prosthetic tissue leaflets can occupy.

6. The method of claim 1, wherein an amount of time between crimping the prosthetic heart valve and deploying the prosthetic heart valve is under 45 minutes.

7. The method of claim 1, wherein an amount of time between crimping the prosthetic heart valve and deploying the prosthetic heart valve is between 2 weeks and 6 months.

8. A method of manufacturing a prosthetic heart valve, the method comprising:
inserting the prosthetic heart valve into a crimper, the prosthetic heart valve including a frame, an inner cuff disposed on a luminal surface of the frame, and a plurality of prosthetic tissue leaflets mounted within the frame, the prosthetic heart valve having an initial diameter when inserted into the crimper;
while the prosthetic heart valve is inserted into the crimper, crimping the prosthetic heart valve onto a delivery device so that the prosthetic heart valve has a crimped diameter that is smaller than the initial diameter, wherein when the prosthetic heart valve has the initial diameter, the prosthetic tissue leaflets are in an uncompressed condition, and when the prosthetic heart valve has the crimped diameter, the prosthetic tissue leaflets are in a compressed condition;
determining a size of the prosthetic tissue leaflets in the uncompressed condition;
determining a crimp magnitude of the prosthetic heart valve after crimping the prosthetic heart valve onto the delivery device, the crimp magnitude being calculated as a ratio of (i) the determined size of the prosthetic tissue leaflets in the uncompressed condition to (ii) a total amount of available size that the prosthetic tissue leaflets can occupy when the prosthetic tissue leaflets are in the compressed condition;
comparing the crimp magnitude to a threshold acceptable crimp magnitude; and
based on the comparison, either:
(i) packaging the delivery device and the prosthetic heart valve while the prosthetic heart valve is crimped onto the delivery device after determining from the comparison that the crimp magnitude does not exceed the threshold acceptable crimp magnitude; or
(ii) discarding the prosthetic heart valve after determining from the comparison that the crimp magnitude does exceed the threshold acceptable crimp magnitude.

9. The method of claim 8, wherein the threshold acceptable crimp magnitude is between 110% and 135%.

10. The method of claim 9, wherein the size of the prosthetic tissue leaflets is a volume of the prosthetic tissue leaflets, and the total amount of available size that the prosthetic tissue leaflets can occupy is a total amount of available volume that the prosthetic tissue leaflets can occupy.

11. The method of claim 10, wherein the total amount of available volume that the prosthetic tissue leaflets can occupy is calculated as a volume of a cylinder prescribed around an outer surface of the frame, less a volume of the frame within the cylinder, less a volume of the inner cuff within the cylinder, less a volume of delivery device components within the cylinder.

12. The method of claim 8, wherein the crimp diameter is a minimum diameter corresponding to a maximum crimping force applied by the crimper.

13. The method of claim 8, wherein the crimp diameter is an equilibrium diameter, the equilibrium diameter being achieved after the prosthetic heart valve recoils from a minimum diameter corresponding to a maximum crimping force applied by the crimper.

14. The method of claim 8, wherein the crimper is an automated or motorized crimper.

15. The method of claim 14, wherein prior to crimping the prosthetic heart valve, a desired crimp diameter is determined based on (i) the determined size of the prosthetic tissue leaflets in the uncompressed condition and (ii) the threshold acceptable crimp magnitude.

16. The method of claim 15, further comprising:
after determining the desired crimp diameter, adjusting settings of the automated or motorized crimper to perform the crimping so that the prosthetic heart valve is crimped to the desired crimp diameter.

17. The method of claim 8, wherein after determining from the comparison that the crimp magnitude does not exceed the threshold acceptable crimp magnitude, the delivery device and the prosthetic heart valve are packaged while the prosthetic heart valve is crimped onto the delivery device.

18. The method of claim 8, wherein after determining from the comparison that the crimp magnitude does exceed the threshold acceptable crimp magnitude, the prosthetic heart valve is discarded.

19. A method of implanting a prosthetic heart valve, the method comprising:
inserting the prosthetic heart valve into a crimper, the prosthetic heart valve including a frame, an inner cuff disposed on a luminal surface of the frame, and a plurality of prosthetic tissue leaflets mounted within the frame, the prosthetic heart valve having an initial diameter when inserted into the crimper;
while the prosthetic heart valve is inserted into the crimper, crimping the prosthetic heart valve onto a delivery device so that the prosthetic heart valve has a crimped diameter that is smaller than the initial diameter, wherein when the prosthetic heart valve has the initial diameter, the prosthetic tissue leaflets are in an uncompressed condition, and when the prosthetic heart valve has the crimped diameter, the prosthetic tissue leaflets are in a compressed condition;
before or after inserting the prosthetic heart valve into the crimper, determining a size of the prosthetic tissue leaflets in the uncompressed condition;
determining a crimp magnitude of the prosthetic heart valve after crimping the prosthetic heart valve onto the delivery device, the crimp magnitude being calculated as a ratio of (i) the determined size of the prosthetic tissue leaflets in the uncompressed condition to (ii) a total amount of available size that the prosthetic tissue leaflets can occupy when the prosthetic tissue leaflets are in the compressed condition;
comparing the crimp magnitude to a threshold acceptable crimp magnitude; and
based on the comparison, either:
(i) implanting the prosthetic heart valve into a patient after determining from the comparison that the crimp magnitude does not exceed the threshold acceptable crimp magnitude; or
(ii) discarding the prosthetic heart valve after determining from the comparison that the crimp magnitude does exceed the threshold acceptable crimp magnitude.

20. The method of claim 19, wherein the threshold acceptable crimp magnitude is between 110% and 300%.
